(19) United States
(12) Patent Application Publication
(10) Pub. No.: US 2025/0195541 A1
(43) Pub. Date: Jun. 19, 2025

(54) Supplement for Cervical Health

(71) Applicant: Claire Anderson, Seattle, WA (US)

(72) Inventor: Claire Anderson, Seattle, WA (US)

(21) Appl. No.: 18/544,923

(22) Filed: Dec. 19, 2023

(51) Int. Cl. A61K 31/575; A23L 33/00; A61K 47/02

(52) U.S. Cl. CPC A61K 31/575; A23L 33/40; A61K 47/02

(57) ABSTRACT

A supplement formulated to support women's cervical health. Active ingredients include: a combination of Beta-Sitosterol and Beta-Sitosterolin-Beta-D-Glucoside; Calcium D-Glucarate; Curcumin; Indole-3-carinbol; Green tea extract; Diindolyimethane; Sulforaphane; Borage oil; a B vitamin complex including B1, B2, Niacin, B5, B6, B12, and folate; vitamin C; beta glucans 1,3 and 1,6; zinc; vitamin E; selenium; and Beta carotene.

### CROSS-REFERENCE TO RELATED APPLICATIONS

This application has no related applications.

### BACKGROUND OF THE INVENTION

The general existence of supplements used to support general health and address specific health conditions is known.

### SUMMARY OF THE INVENTION

The instant invention is a novel supplement designed to support cervical health, which has not previously been available as a single combined product with the amounts selected for cervical health. Active ingredients include at least: Beta-Sitosterol and Beta-Sitosterolin-Beta-D-Glucoside, Calcium D-Glucarate, Curcumin, Indole-3-carinbol, Green tea extract, Diindolylmethane, Sulforaphane, Borage oil, B vitamins including B1, B2, Niacin, B5, B6, B12, and folate, vitamin C, beta glucans 1,3 and 1,6, zinc, vitamin E, selenium, and Beta carotene.

### DETAILED DESCRIPTION OF THE INVENTION

In the following description, numerous specific details are set forth to clearly describe the embodiments disclosed herein. However, one skilled in the art will understand that some well known features have not been described in detail so as not to obscure the invention.

A single supplement, formulated to improve the cervical health of women.

Active ingredients of the supplement include the following:
A combination of Beta-Sitosterol and Beta-Sitosterolin-Beta-D-Clucoside, currently available within the separate supplement known as Moducare, 20 mg.
Calcium D-glucarate, 300 mg.
Curcumin, minimum concentration 95%, 100 mg.
Indole-3-carinbol, 300 mg.
Green tea extract, 200 mg.
Diindolylmethane, 100 mg.
Sulforaphane, 400 mcg.
Borage oil, 2000 mg.
B vitamins, including 100 mg of B1, 7.5 mg of B2, 353 mg of Niacin, 300 mg of B5, 100 mg of B6, 1000 mcg of B12, and 1000 mcg of folate.
Vitamin C, 1000 mg.
Beta glucans 1,3 and 1,6 250 mg.
Zinc, 30 mg.
Vitamin E, 200 mg.
Selenium, 200 mcg.
Beta carotene, 7,500 mcg.

It is explicitly within the scope of the invention that known, similarly bioactive substitutions may be used as the active ingredients, as does the use of variants with other names for the same chemicals. Vitamin B1 is also known as thiamin; B2 is riboflavin; niacin has been called both vitamin B3 and nicotinic acid; vitamin B5 is pantothenic acid; vitamin B6 is pyridoxine; vitamin B12 is cobalamin; and folate is also known as vitamin B9. Although folate itself is not chemically identical to folic acid, and is somewhat less bioavailable for at least some people, the use of folic acid for the purpose of folate supplementation within foods and supplements is known and explicitly included within the scope of this disclosure as an alternate embodiment, which is not the preferred embodiment due to the reduced bioavailability. Similarly, other forms of calcium are explicitly included within the scope of this disclosure, as alternate and less preferred embodiments. Any other active ingredient with known chemical variants which are biologically active such that they offer similar efficacy as used in this novel combination of active ingredients is considered to be included within the scope of this disclosure.

Although each of the active ingredients may be found within individual or combination dietary supplements, the combination of active ingredients is not readily available, and they have a synergistic effect when taken together, resulting in a greater impact than from separate supplementation. For example, calcium and zinc are commonly found in a common supplement with vitamin D and magnesium because the body uses them to process each other, so what appears to be a deficiency in one may be best remedied by also consuming the others. Although some of the disclosed and recited active ingredients have been shown to be beneficial to cervical health individually or in sub-combinations, the total combination of active ingredients and their proportions has not been available and is novel.

This disclosure also explicitly includes various fillers, binders, and coatings for dietary supplements, as are known in the art.

While illustrative embodiments of the invention have been shown and described, variations and alternative embodiments will occur to those skilled in the art. Such variations and alternative embodiments can be made without departing from the scope of the invention as defined in the claims.

As used in this specification and the appended claims, the singular forms “a” and “an” indicate a single element, while “the” may refer back to single or plural referents. Unless defined otherwise, all technical and scientific terms used herein have the same meaning as commonly understood by one of ordinary skill in the art to which the disclosure pertains.

The above detailed description of exemplary and preferred embodiments is presented for the purposes of illustration and disclosure in accordance with the requirements of the law. It is intended to be exemplary but not exhaustive, and is not intended to limit the invention to the precise forms described, but only to enable others skilled in the art to understand how the invention may be suited for a particular use of implementation. No limitation is intended by the description of exemplary embodiments which may have included tolerances, feature dimensions, specific operating conditions, engineering specifications, or the like, and which may vary between implementations or with changes to the state of the art, and no such limitation should be implied therefrom. Applicant has made this disclosure with respect to the current state of the art, but also contemplates advancements and that adaptations in the future may take into consideration those advancements in accordance with the then current state of the art. It is intended that the scope of the invention be defined by the Claims as written and equivalents as applicable. Reference to a claim element in the singular is not intended to mean “one and only one” unless explicitly so stated. No claim element herein is intended to be construed under the provisions of 35 U.S.C. 112(f), unless the element is expressly recited using the exact phrase “means for . . . ” and no method or process step herein is to be construed under the provisions of 35 U.S.C. section 112(f) unless the step, or steps, are expressly recited using the exact phrase “step(s) for . . . ”.

1. A supplement for cervical health, with active ingredients comprising: Beta-sitosterol, Beta-sitosterolin-Beta-D-Glucoside, Calcium D-glucarate, Curcumin, Indole-3-carinbol, Green tea extract, Diindolylmethane, Sulforaphane, Borage oil, B vitamins, Vitamin C, Beta glucans 1,3 and 1,6, zinc, vitamin E, selenium, and beta carotene.

2. The supplement for cervical health of claim 1, wherein the B vitamins comprise B1, B2, Niacin, B5, B6, B12, and folate.

3. The supplement of claim 1, wherein the Beta-Sitosterol and Beta-Sitosterolin-Beta-D-Glucoside combine to a mass of approximately 20 mg per serving.

4. The supplement of claim 1, wherein the curcumin has at least 95% purity and is included in an amount of approximately 100 mg per serving.

5. The supplement of claim 1, wherein the indole-3-carinbol has a mass of approximately 300 mg per serving.

6. The supplement of claim 1, wherein the green tea extract has a mass of approximately 200 mg.

7. The supplement of claim 1, wherein the diindolylmethane has a mass of approximately 100 mg per serving.

8. The supplement of claim 1, wherein the sulforaphane has a mass of approximately 400 mcg per serving.

9. The supplement of claim 1, wherein the borage oil is present in an amount of approximately 2000 mg per serving.

10. The supplement of claim 1, wherein each serving includes approximately 100 mg of B1, 7.5 mg of B2, 353 mg of Niacin, 300 mg of B5, 100 mg of B6, 1000 mcg of B12, and 1000 mcg of folate.

11. The supplement of claim 1, wherein each serving includes approximately 1000 mg of vitamin C.

12. The supplement of claim 1, wherein the beta glucans 1,3 and 1,6 combine to a mass of approximately 250 mg per serving.

13. The supplement of claim 1, wherein zinc is present in an amount approximately 30 mg per serving.

14. The supplement of claim 1, wherein vitamin E is present in the amount of approximately 200 mg per serving.

15. The supplement of claim 1, wherein selenium is present in the amount of approximately 200 mcg per serving.

16. The supplement of claim 1, wherein beta carotene is present in the amount of approximately 7,500 mcg per serving.
